(19) United States
(12) Patent Application Publication
(10) Pub. No.: US 2013/0217681 A1
(43) Pub. Date: Aug. 22, 2013

(54) METHOD AND COMPOSITION FOR WEIGHT-GAIN MANAGEMENT

(71) Applicants: Avraham Weizman, Tel Aviv (IL); Michael Poyurovsky, Haifa (IL)

(72) Inventors: Avraham Weizman, Tel Aviv (IL); Michael Poyurovsky, Haifa (IL)

(73) Assignees: TECHNION RESEARCH AND DEVELOPMENT FOUNDATION LTD., Haifa (IL); RAMOT AT TEL-AVIV UNIVERSITY LTD., Tel Aviv (IL)

(21) Appl. No.: 13/883,158

(22) PCT Filed: Nov. 22, 2011
(86) PCT No.: PCT/IL11/50021

§ 371 Date: May 2, 2013

Related U.S. Application Data
(60) Provisional application No. 61/417,401, filed on Nov. 26, 2010.

(51) Int. Cl. A61K 31/5375; A61K 31/4402; A61K 31/5513

(52) U.S. Cl. CPC A61K 31/5375; A61K 31/5513; A61K 31/4402
USPC 514/220; 514/239.2

(57) ABSTRACT

Presented herein is a methodology for reducing weight in obese subjects and in patients receiving various medical treatments that are accompanied with weight gain. The methodology allows for management of weight gain, management of triglyceride levels and weight reduction in obese subjects.

### FIELD OF THE INVENTION

This invention generally relates to a method and composition for reducing weight and/or controlling weight-gain in obese subjects and in patients receiving medical treatment that is accompanied with weight gain.

### BACKGROUND OF THE INVENTION

Obesity, in which excess body fat is gained to the extent that it may have an adverse effect on health, affects a substantial proportion of children and adults worldwide (˜25% in the United States), and is associated with a wide range of metabolic and cardiovascular conditions, such as diabetes and heart attacks. Diet and exercise are of some value, but many individuals are not able to maintain moderate weight loss with diet and exercise alone.

Weight gain in human individuals can be attributed to various reasons, including a lack of exercise due to fatigue, hormonal treatments, steroids and pain medications, and eating more than one requires as a way to cope at a stressful time. In addition, weight gain has been recognized to accompany various medical treatments, such as treatment of hyperthyroidism [1].

Triglycerides are the primary fat in our bodies and the main constituent in our energy system. The risk of having a heart attack or stroke is especially high when triglycerides accompany obesity and high cholesterol. In addition, triglycerides may block a hormone that controls appetite and calorie burning. Obesity has a direct causal link with high levels of triglycerides such that there is a significant correlation between obesity and plasma triglycerides levels, with heavier individuals having higher triglyceride levels. Thus, the power to control plasma triglyceride levels is desirable for controlling weight gain.

A consistent adverse effect of atypical antipsychotic agents is weight gain. Olanzapine is an efficacious antipsychotic medication, but like other atypical agents, olanzapine has been show to induce significant weight gain in patients treated with this drug (e.g. 3.5 kg in 10 weeks; [2]).

Development of successful treatment of obesity has been limited, in part because of attempts to deal with complex phenomena of obesity by targeting a single pathophysiological mechanism. Research on CNS pathways that regulate food intake and bodyweight has identified a complex interplay between multiple neurotransmitter systems, hormones and peptides. Combinations of drugs that target multiple systems are common in the treatment of diabetes, hypertension and other complex disorders, and it was suggested that such an approach might also be more effective than monotherapy in the treatment of obesity. Indeed, recently, positive results have been reported for late-stage clinical trials evaluating an anti-obesity effect of numerous combination products such as Qnexa (topiramate and phentermine, Vivus Pharmaceuticals), Contrave (bupropion and naltrexone, Orexigen Pharmaceuticals) and Empatic (bupropion and zonizamide, Orexigen Pharmaceuticals) [3, 4]. These combinations include agents that act through distinct weight control mechanisms. They have been demonstrated to cause weight loss while treating other conditions, while being administered in lower doses as compared to doses typically prescribed as individual components. Although the administered combinations of drugs are promising in terms of efficacy, their safety profile is far from benign. For example, bupropion has been associated with seizures and exacerbation of psychosis when used in psychiatric patients to counteract antipsychotic-drug induced weight gain and topiramate has been linked to an increased risk of suicide, as well as cognitive side effects such as memory impairment and confusion, teratogenicity, and rare cases of glaucoma. Hence, a further search for effective, safe and tolerable anti-obesity treatment is warranted.

The noradrenergic system has consistently been implicated in regulation of food intake and body weight and agents that facilitate adrenergic neurotransmission have been shown to be potent appetite-suppressants (e.g., phentermine, amphetamine). Noradrenergic (NE) drugs available in the United States include phentermine, diethylpropion, phendimetrazine and benzphetamine. Their major drawback, however, is a potential for abuse. Amphetamines which are considered to have a particularly high potential for abuse are no longer recommended for weight loss for this reason. Reboxetine, an antidepressant drug used in the treatment of clinical depression, panic disorder and ADD/ADHD, has been demonstrated to increase adrenergic tone by a selective blockade of the noradrenergic transporter. It was reported that a low-dose of reboxetine (4 mg/day) exerts a significant weight-attenuating effect in schizophrenic patients treated with olanzapine, an antipsychotic with the highest weight-gain potential among antipsychotic agents [5, 6]. In contrast to sibutramine and bupropion, the tolerability of reboxetine was determined superior; it was safe and well tolerated and no clinically significant side effects were found in reboxetine-olanzapine treated patients. In addition, reboxetine did not interfere with olanzapine's antipsychotic effect.

Betahistine is a structured analog of histamine that exerts H₁ receptor agonistic and H₃ receptor antagonistic properties [7, 8]. The drug crosses the blood-brain barrier and acts centrally by enhancing histamine synthesis in tuberomammillary nuclei of the posterior hypothalamus. Betahistine has been shown to inhibit food intake and increase the satiety signal in animal models of obesity [9, 10]. It was reported that betahistine is safe, well tolerated and prevents weight gain in olanzapine-treated first episode schizophrenia patients, to a certain degree [11]. A major limitation of this study was a small sample size and open design.

### REFERENCES

[1] Abid M, et al., 1999. Journal of the American College of Nutrition, Vol. 18, No. 2, 189-193

[2] Allison et al., 1999. Am J Psychiatry 156: 1686-1696.

[3] Greenway F L, et al., 2010. Lancet; 376(9741):595-605.

[4] Roth et al., 2010., Brain Res; 1350:86-94.

[5] Poyurovsky M, et al., 2003. Am J Psychiatry; 160(2):297-302.

[6] Poyurovsky M, et al., 2007. Psychopharmacology (Berl); 192(3):441-8.

[7] Fossati A, et al 2001. Pharmacol Res; 43(4):389-92.

[8] Lacour M, et al., 2001. CNS Drugs; 15(11):853-70.

[9] Szelag A, et al., 2001. Pol J Pharmacol; 53(6):701-7.

[10] Rossi R, et al., 1999. Physiol Behav; 66(3):517-21.

[11] Poyurovsky M, et al., 2005. Int Clin Psychopharmacol; 20(2):101-3.

### SUMMARY OF THE INVENTION

The increasing incidence of obesity is a recognized medical problem in developed countries. Obesity is a major factor for a number of diseases, including non-insulin dependent diabetes mellitus, coronary heart diseases, hypertension, pulmonary dysfunction and even certain types of cancer. Treatment of obesity is beneficial in that weight loss reduces the risk for mortality and morbidity such that even modest weight loss results in beneficial health effects.

In an attempt to reduce weight in obese subjects and in patients receiving various medical treatments that are accompanied with weight gain, the inventors of the present invention have developed a combination therapy for management of weight gain, management of triglyceride levels and for weight reduction in obese subjects.

Thus, in one of its aspects, the present invention provides a composition comprising at least one H₃-receptor antagonist and at least one norepinephrine reuptake inhibitor (NRI).

In some embodiments, the composition is a pharmaceutical composition.

In other embodiments, the invention provides a composition for controlling weight-gain (e g , minimizing or preventing weight gain), the composition comprising at least one H₃-receptor antagonist and at least one norepinephrine reuptake inhibitor (NRI).

As used herein, the “H₃-receptor antagonist” refers to a drug used to block the action of histamine at the H₃-receptor primarily found in the brain. The H₃-receptor antagonist, as defined herein, is effective to contribute to at least one of the following: a reduction in weight, a reduction and/or control of weight gain and suppression of appetite—in obese as well as non-obese subjects and in subjects undergoing medical treatment which is accompanied by weight gain (e.g. the treatment of the psychiatric disease or disorder in a patient, as described herein). In accordance with the present invention, the H₃-receptor antagonist may also have at least a partial H₁-receptor agonistic activity, e.g., a direct stimulating (agonistic) effect on H₁-receptors located on blood vessels in the inner ear). Some non-limiting examples of H₃-receptor antagonist include betahistine hydrochloride, betahistine mesylate, R-α-methyl histamine, methimepip, ciproxifan and thioperamide

The “norepinephrine reuptake inhibitor” (NRI) refers to a drug which acts as a reuptake inhibitor for the neurotransmitters norepinephrine (noradrenaline) and epinephrine (adrenaline) by blocking the action of the norepinephrine transporter. In accordance with the present invention, the NRI is effective to contribute to at least one of the following: a reduction in weight, a reduction and/or control of weight gain and suppression of appetite—in obese as well as non-obese subjects and in subjects undergoing medical treatment which is accompanied by weight gain (e.g. the treatment of the psychiatric disease or disorder in a patient, as described herein). Some non-limiting examples of NRIs that can be used in accordance with the present invention include Serotonin-Norepinephrine-Dopamine Reuptake Inhibitors, Selective Norepinephrine Reuptake Inhibitors (SNRI), Norepinephrine-Dopamine Reuptake Inhibitors (NDRIs), Serotonin-Norepinephrine Reuptake Inhibitors (SNRIs), Tricyclic Antidepressants (TCAs), and Tetracyclic Antidepressants (TeCAs). In some embodiments, the NRI is an SNRI selected form atomoxetine/tomoxetine; mazindol; reboxetine and viloxazine.

As used herein, “controlling weight-gain” refers to any one or more of preventing, arresting and reducing weight-gain, whereby at least one or more of the following is achieved:

(i) decrease in body fat or body weight;

(ii) decrease or maintenance of plasma triglyceride levels;

(iii) prevention of weight gain and/or cessation of weight gain;

(iv) improvement in leptin resistance;

(v) reduction in hyperglycemia and/or decrease in incidence or severity of diabetes;

(vi) reduction in hyperlipidaemia and/or hypertriglyceridemia;

(vii) decrease in food intake;

(viii) improvement in at least one condition associated with weight gain including a muscosceletal disorder, a cardiovascular disorder, a dermatological disorder, a sleep disorder, a metabolic condition, a diabetes-related condition;

(ix) at least partial improvement (e.g., termination of or reduction in occurrence) of a condition selected from binge eating disorder, night eating syndrome, obsessive eating, compulsive eating or bulimia; and

(x) compliance of a human subject to caloric restriction. In accordance with some embodiments of the present invention the subject is an obese subject.

In some embodiments, the composition for controlling weight-gain is used for achieving a weight reduction in obese subjects. In other embodiments, the composition for controlling weight-gain is used for the suppression of appetite. In still other embodiments, the composition for controlling weight-gain is used for controlling, as further defined below, weight gain in patients, e.g., psychiatric patients, who are treated with a drug, e.g., a psychiatric drug, which use is associated with weight gain.

Thus, the invention also provides a composition for inducing weight reduction in an obese subject, the composition comprising at least one H₃-receptor antagonist and at least one norepinephrine reuptake inhibitor (NRI).

The invention also contemplates a composition for reducing or maintaining plasma triglyceride levels in obese subjects and also in subjects of normal weight in need of a reduction and/or maintenance of plasma triglyceride levels (e.g. subjects receiving medication which side effect is an increase in plasma triglyceride levels), the composition comprising at least one H₃-receptor antagonist and at least one norepinephrine reuptake inhibitor (NRI).

An “obese” subject, human or non-human, is one which suffers from a medical condition in which excess body fat has accumulated to the extent that it may have an adverse effect on health. Typically, obesity is defined by parameters such as body mass index (BMI) (e.g. according to the American Obesity Association an adult human having a Body Mass Index (MBI) over 25 is considered to be obese), fat distribution via the waist-hip ratio, elevated plasma triglyceride levels and various additional parameters as recognized by the skilled artesian.

“Weight reduction”, thus, refers to achieving a weight reduction in the obese subject, administered with the herein defined composition, of at least 1, 2, 3, 5, 10 or even 15% of the body weight (as measured prior to the administration of said composition to the subject, i.e., baseline body weight). In the context of the present invention, weight reduction encompasses also the maintenance of a subject's weight and also the maintenance of plasma triglyceride levels achieved after weight reduction. A weight reduction may be considered maintained when at least 1% of the initial body weight has been reduced and the reduction is maintained for at least 3 months after initial administration of the herein defined composition.

Controlled diet and controlled appetite is one of the most prevalent techniques for controlling obesity. Thus, in another one of its aspects the present invention provides a composition for the suppression of appetite in a subject, the composition comprising at least one H₃-receptor antagonist and at least one NRI.

“Appetite suppression” or any lingual variation thereof (interchangeable with ‘satiety induction’), refers, generally, to an increase in the feeling of ‘fullness’ (i.e., prolonged satiety) in subjects being administered with the herein defined composition, resulting in reduced food (caloric) intake during a meal and/or between meals. For the herein defined purposes, the satiety inducing composition may be admixed with food in an amount effective to extend the satiation effect of that food, and achieve appetite suppression and a reduction in caloric intake.

In some embodiments, the appetite suppressant effect is prolonged for at least three hours after a meal. In other embodiments, the appetite suppressant effect is prolonged for at least six hours after a meal.

In accordance with the present invention, the appetite suppressant effect typically results in a reduction in body weight and/or plasma triglyceride levels, and, in some embodiments, also in the maintenance of this reduction for time periods of at least a few weeks (e.g., 4, 6, 8 weeks or more) or a few months (e.g., 4, 6, 8 months or more) and even a few years from the initial administration of the composition of the invention.

In some embodiments, the subject (obese or non-obese) has at least one eating disorder, i.e., a condition characterized by abnormal eating habits that may involve excessive food intake to the detriment of an individual's physical and emotional health. Some non-limiting examples of such eating disorders are binge eating disorder (BED), nocturnal sleep related eating disorder (NSRE) and bulimia nervosa (BN).

In accordance with some embodiments of the present invention, the subject is a subject being treated with at least one drug. In some embodiments, the treatment with the at least one drug is accompanied with weight gain and/or elevated plasma triglyceride levels (e.g. 200 to 1000 mg/dL) in the subject, being a side effect of the treatment with the at least one drug.

In some other embodiments, the subject is treated with at least one psychiatric drug wherein the treatment is accompanied with weight gain and/or elevated plasma triglyceride levels in the subject.

Thus, the present invention is also directed to a composition for controlling weight-gain in a subject being treated with a psychiatric drug, the composition comprising at least one H₃-receptor antagonist, as defined herein, and at least one NRI, as defined herein.

In another one of its aspects the present invention provides a composition for treating a psychiatric disease or disorder in a subject, without substantially inducing weight gain and/or without substantially increasing plasma triglyceride levels in said subject, the composition comprising at least one psychiatric drug, at least one H₃-receptor antagonist and at least one norepinephrine reuptake inhibitor (NRI).

The present invention is also directed to a composition for reducing or maintaining plasma triglyceride levels in a subject being treated with a psychiatric drug, the composition comprising at least one H₃-receptor antagonist, as defined herein, and at least one NRI, as defined herein.

As used herein, the “psychiatric drug” is a drug which is commonly used for the treatment of psychiatric diseases or disorders. The psychiatric drug employed is selected to be effective in reducing at least one psychiatric symptom associated with the psychiatric disease or disorder, in a patient suffering therefrom or has a predisposition to suffer therefrom and for whom such treatment is intended. The psychiatric drug is selected amongst drugs that are effective against both positive symptoms of the psychiatric disease or disorder, i.e., hallucinations, delusions and racing thoughts, as well against negative symptoms, i.e., apathy, lack of emotion and poor or nonexistent social functioning.

In some embodiments, the psychiatric drug is an atypical antipsychotic (AAP; also known as ‘second generation antipsychotics’), namely an antipsychotic tranquilizing drugs used in the treatment of psychiatric conditions. Some non-limiting examples of atypical antipsychotics include amisulpride, aripiprazole, asenapine, blonanserin, clotiapine, clozapine, iloperidone, mosapramine, olanzapine, paliperidone, perospirone, quetiapine, remoxipride, risperidone, sertindole, sulpiride, ziprasidone and zotepine.

In some embodiments, the at least one drug is olanzapine.

In some embodiments, the composition of the invention comprises at least one H₃-receptor antagonist and reboxetine. In further embodiments, the composition comprises at least one NRI and betahistine and in still additional embodiments, the composition of the invention comprises betahistine and reboxetine.

In some embodiments, the composition of the invention comprises at least one NRI, at least one H₃-receptor antagonist and olanzapine. In other embodiments, the composition of the invention comprises reboxetine, at least one H₃-receptor antagonist and at least one psychiatric drug. In further embodiments, the composition of the invention comprises at least one NRI, betahistine and at least one psychiatric drug.

In further embodiments, the composition of the invention comprises betahistine, reboxetine and at least one psychiatric drug and in still further embodiments, the composition of the invention comprises betahistine, reboxetine and olanzapine.

While it is possible for the herein disclosed compositions to be administered per se without the addition of a carrier, it is preferable to administer the compositions of the invention as pharmaceutical formulations which further comprise at least one carrier, excipient or diluent.

Thus, the present invention also provides pharmaceutical formulations comprising the herein defined combinations together with one or more pharmaceutically acceptable carriers or excipients and optionally other therapeutic agents. The formulation can be administered by any acceptable route, e.g., oral in any acceptable form, (e.g., a tablet, a capsule, a solution, a liquid, a suspension, or an emulsion). The carrier(s) must be acceptable in the sense of being compatible with the other ingredients of the formulation and not harmful to the recipient thereof. When the individual components of the combination (e.g., H₃ receptor antagonist, NRI or psychiatric drug) are administered separately they are usually each presented as a pharmaceutical formulation. Thus, for the purposes of the present invention, either the herein defined combinations or pharmaceutical formulations comprising the combinations (or any part thereof) can be used.

The pharmaceutical compositions of the present invention may be administered by any suitable route for the delivery of the specific composition to the subject to be treated. In some embodiments, the route of administration is selected from oral, transdermal, intravenous, subcutaneous, intramuscular, intranasal, intraauricular, sublingual, rectal, transmucosal, intestinal, buccal, intramedullar, intrathecal, direct intraventricular, intraperitoneal and intraocular routes.

In accordance with the present invention, the compositions of the invention, as disclosed herein, can be used in simultaneous, separate or sequential medical therapy. Accordingly, the individual components of any of the compositions of the invention (i.e., H₃-receptor antagonist, NRI or psychiatric drug) or any combination thereof can be administered simultaneously, separately or sequentially, to a patient in need thereof.

As used herein, “simultaneously” or any lingual variation thereof is used to mean that the components of a composition are administered concurrently, e.g., one after the other. Simultaneous administration may permit one component in the combination to be administered within a certain time period (e.g., 5 minutes, 10 minutes or even a few hours) after the other, provided that the circulatory half-life concentration of the first administered component in a combination is concurrently present in therapeutically effective amounts with the other components administered thereafter. The time delay between administration of the components may vary depending on the exact nature of the components and the formulation containing them, the interaction between the individual components, their respective half-lives, and on such other factors as easily recognized by the versed artesian.

In contrast, “sequentially” or “separately” or any lingual variation thereof is used herein to mean that the time period between administering one component and the other is significant i.e., the first administered component may no longer be present (or is present in subclinical amounts) in the bloodstream in a therapeutically effective amount when the second (or subsequent) component is administered.

Administration of a combination according to the invention results in an improved weight reducing effect, in obese subject or in patients treated with a drug that causes weight gain (e.g. psychiatric patients treated with at least one psychiatric drug, alone or as part of the composition), as compared to the administration of either component (i.e., H₃-receptor antagonist or NRI) alone and permits dosing of each component at a dosage significantly lower than would be required to obtain beneficial effects from either components if it were to be administered separately from the other. Without wishing to be bound by theory, the effectiveness of the compositions in reducing weight gain is believed to result from the involvement of two different biological cascades—one associated with the blocking the action of the norepinephrine transporter and the other with the deactivation of the H₃-receptor. Thus, in accordance with some embodiments of the invention, the herein defined combinations are synergistic for each of the defined purposes (e.g. weight reduction, reduction or maintenance of plasma triglyceride levels, appetite suppression, control of weight gain).

In another one of its aspects, the present invention provides a method for controlling weight gain in a subject, the method comprising administering to said subject a combination of at least one H₃-receptor antagonist and at least one norepinephrine reuptake inhibitor (NRI).

In another one of its aspects, the present invention provides a method for weight reduction in obese subjects, the method comprising administering to said subject a combination of at least one H₃-receptor antagonist and at least one norepinephrine reuptake inhibitor (NRI).

In still another one of its aspects, the present invention provides a method for the suppression of appetite in a subject, the method comprising administering to said subject a combination of at least one H₃-receptor antagonist and at least one norepinephrine reuptake inhibitor (NRI).

In an additional aspect, the present invention provides a method for controlling weight-gain in a subject being treated with a psychiatric drug, the method comprising administering to said subject at least one H₃-receptor antagonist and at least one norepinephrine reuptake inhibitor (NRI).

In a further aspect, the present invention provides a method for treating a psychiatric disease or disorder in a subject, without substantially inducing weight gain and/or without substantially increasing plasma triglyceride levels in said subject, the method comprising administering to said subject a combination of at least one psychiatric drug, at least one H₃-receptor antagonist and at least one norepinephrine reuptake inhibitor (NRI).

The present invention is also directed to a method for reducing or maintaining plasma triglyceride levels in a subject, the method comprising administering to said subject a combination of at least one H₃-receptor antagonist and at least one norepinephrine reuptake inhibitor (NRI). In some embodiments, the subject is a subject being treated with a psychiatric drug, as described herein.

As used herein, “treating” a psychiatric disease or disorder refers to any amelioration of one or more symptoms of the psychiatric disease or disorder. The term also encompasses the prophylaxis of a physical and/or mental condition associated with the psychiatric disease or disorder or amelioration or elimination of the developed physical and/or mental condition once it has been established or alleviation of the characteristic symptoms of such condition.

As used herein, the “psychiatric disease or disorder” is any pathological psychological disease or disorder (as characterized in the DSM-IV-TR. Diagnostic and Statistical Manual of Mental Disorders, Revised, 4rd Ed., 2000), which is treatable by said at least one psychiatric drug used in a composition or method according to the present invention. It is to be understood that when a psychiatric disease or disorder is treated over a short period of time, weight-gain may be minimal and reversible once treatment with the psychiatric drug is discontinued. However, with long-term treatment, patients treated with at least one psychiatric drug, as described herein, may gain a significant amount of weight, possibly reducing their readiness to comply with treatment.

In some embodiments, according to the invention, the psychiatric disease or disorder is psychosis, namely an abnormal condition of the mind that is often described as involving a loss of contact with reality. Typically, psychosis refers to severe forms of psychiatric disorder, during which hallucinations and delusions and impaired insight may occur. A psychosis can be associated with or can evolve from functional causes such as: brain tumors; drug abuse amphetamines, cocaine, alcohol among others; brain damage; schizophrenia, schizophreniform disorder, schizoaffective disorder, brief psychotic disorder; bipolar disorder (manic phase and depression phase); severe clinical depression; severe psychosocial stress; sleep deprivation; some focal epileptic disorders especially if the temporal lobe is affected; exposure to some traumatic event (violent death, etc.) and abrupt or over-rapid withdrawal from certain recreational or prescribed drugs.

In some embodiments, the psychosis is selected from psychotic depression, bipolar mania, bipolar depression, schizophrenia, a schizoaffective disorder, a schizophreniform disorder, a brief psychotic disorder (i.e., psychotic symptoms that last between 1 and 30 days), a delusional disorder, a shared psychotic disorder, a substance-induced psychotic disorder, a psychotic disorder due to a medical condition and paraphrenia, and dopamine agonist-induced psychosis in Parkinson's disease patients.

Where the composition of the invention comprises also the psychiatric drug, H₃-receptor antagonist and NRI, the components may be administered simultaneously, either in the same or in different pharmaceutical formulations, or sequentially at different time points. When the components are administered sequentially, the delay between the administration of one component and other components of the composition should be such as not to lose the benefit of the efficacious effects of the composition as a whole. Thus, for example, the treatment may begin by simultaneously co-administering all three components of a composition according to the invention to a patient suffering from a certain psychiatric disease or disorder whereby the patient receives a daily, twice daily, three times daily etc. dose comprising all three components in a dosing regimen dictated by the medical practitioner treating the patient. In accordance with such embodiments, all three components of the composition may be administered in a single pharmaceutical formulation or in separate pharmaceutical formulations depending on various parameters, such as the bioavailability of the various components, their pharmacokinetic properties, the type of carrier present in each formulation, the physical condition of the patient, the type of the psychiatric disease and so on, as recognized by the person of skill in the art.

In some embodiments, the subject treated in accordance with the invention is first administered with the psychiatric drug for a certain time period depending on the patient's response to the drug. Depending on the weight gained by the patient (as well as on various other parameters such as glucose and lipid blood levels, e.g. plasma triglyceride levels) as a result of treatment with the drug, the patient is administered with one or two of H₃-receptor antagonist and NRI, wherein the administration regimen of the H₃-receptor antagonist and/or NRI depends on the amount of weight gained by the patient since initiation of treatment with the psychiatric drug and on various other parameters, such as conditions associated with obesity (e.g. plasma triglyceride levels) as well as the extent of side effects and drug interactions, as recognized by the skilled artesian.

It is to be understood that, in accordance with the present invention, the amount of each of the components of the herein defined combinations (or components thereof) required to produce an efficacious effect will vary and is ultimately at the discretion of the medical practitioner. The factors to be considered include the route of administration and nature of the formulation, the patient's body weight, plasma triglyceride levels, age and general condition (and the nature and severity of the psychiatric disease to be treated wherein the subject is a subject having a psychiatric disease).

In some embodiments, when a patient is under chronic use of a psychiatric drug, or any other drug including non-psychiatric drugs, which use results in a significant weight-gain and metabolic side effects associated therewith (such as hyperglycemia and hyperlipidaemia) the patient may be administered with the H₃-receptor antagonist and NRI combination for a limited time period, for example during weeks or months in which the patient gains more weight as compared to other periods. As an illustrative example, the medical practitioner may decide that when the patient gains more than e.g., 2kg per month for a time period exceeding, e.g., 2 months, treatment with the aforementioned combination may be initiated until the gain in weight decreases to below, e.g., 0.5 kg/month.

In some embodiments, the combination comprising H₃-receptor antagonist and NRI is administered to a patient being treated with the at least one psychiatric drug (or any other drug including non-psychiatric drugs) at the beginning of the treatment. In other embodiments, the combination comprising H₃-receptor antagonist and NRI is administered only when the patient, being treated with at least one psychiatric drug (or any other drug including non-psychiatric drugs), gains weight, exhibits higher than normal plasma triglyceride levels and/or is detected to suffer from at least one obesity related condition (e.g. hyperglycemia, hyperlipidaemia).

In still another one of its aspects the present invention provides the use of at least one H₃-receptor antagonist and at least one NRI for preparing a medicament for controlling weight-gain in a subject.

In still yet another one of its aspects, the present invention provides the use of at least one H₃-receptor antagonist and at least one NRI for preparing a medicament for reducing or maintaining plasma triglyceride levels in a subject. In some embodiments the subject is a subject being treated with a psychiatric drug, as described herein. The present invention is also directed to the use of at least one H₃-receptor antagonist and at least one NRI for preparing a medicament for reducing weight in obese subjects.

In another one of its aspects the present invention provides the use of at least one H₃-receptor antagonist and at least one NRI for preparing a medicament for suppressing appetite in a subject.

The present invention also provides the use of at least one H₃-receptor antagonist and at least one NRI for preparing a medicament for controlling weight gain in a subject being treated with a psychiatric drug, said weight gain being associated with said treatment with the psychiatric drug.

In still yet another one of its aspects the present invention provides the use of at least one psychiatric drug, at least one H₃-receptor antagonist and at least one NRI for preparing a medicament for treating a psychiatric disease or disorder in a subject, without substantially inducing weight-gain and/or without substantially increasing plasma triglyceride levels in said subject.

The present invention is also directed to kits and commercial packages comprising the compositions of the invention. The components composed in any of the kits of the invention, may be contained in a single vessel or holding unit or in separate vessels. The kit form is particularly advantageous when the components are contained in different vessels for administration in different dosage amounts or when titration of the individual components of the combination (e.g., H₃-receptor antagonist and NRI or H₃-receptor antagonist, NRI and psychiatric drug) is desired by the prescribing physician.

Various embodiments and aspects of the present invention as delineated hereinabove and as claimed in the claims section below find experimental support in the following examples.

### DETAILED DESCRIPTION OF EMBODIMENTS

### Method

Human patients (male and female, age 18-45) hospitalized at Tirat Carmel Mental Health Center (Tirat Carmel, Israel) were screened. Eligible patients were interviewed using the Structured Clinical Interview for DSM-IV-TR Axis I Disorders, Patient Edition, and those who meet DSM-IV-TR criteria for schizophrenic or schizophreniform disorder were enrolled.

Inclusion criteria for the study were:
1) indication for olanzapine treatment during the current hospitalization,
2) no evidence of previous olanzapine exposure;
3) less than four weeks of antipsychotic drug treatment in the preceding six months prior to hospitalization; and
4) patients' competence to provide written informed consent.

Exclusion criteria were:
1) major mood disorders (MDD or bipolar disorder), drug or alcohol abuse and dependence; organic brain syndrome;
2) unstable physical or endocrine disease;
3) a personal or family history of diabetes mellitus; and
4) body mass index (BMI) >30 kg/m².

A double-blind, placebo-controlled, randomized design was used in the study. All participants will be allocated to receive a fixed dose of olanzapine (10 mg at 8:00 p.m.) with either betahistine (48 mg/day [16mg t.i.d.]) and reboxetine 4 mg or placebo for 6 weeks.

The doses of all psychotropic medications remained unchanged during the entire study period.

Meals were served three times a day, and patients were not placed on a special diet or physical exercise program for weight reduction.

### Results

### Example 1

Thirty non-obese olanzapine-treated schizophrenia patients (24 men, 6 women; age 21-50 years) were co-administered the combination of reboxetine and betahistine for the duration of 6 weeks. Six patients discontinued the study due to reasons unrelated to the study medication. Twenty four patients completed the trial. The control group included 13 patients (11 men, 2 women, age 20-40 years) treated with olanzapine co-administered with placebo. Table 1 depicts body weight and increase in body weight each week from baseline in the two study groups.

TABLE 1
body weight and increase in body weight from onset of study.

| | Combination group | | Placebo group | |
|---|---|---|---|---|
| | (olanzapine + reboxetine/betahistine) | | (olanzapine + placebo) | |
| | N = 30 | | N = 13 | |
| | | Δ from | | Δ from |
| Baseline | 69.1 kg | baseline | 69.1 Kg | baseline |
| Week 1 | 70.4 Kg | 1.3 kg | 70.4 Kg | 1.2 kg |
| Week 2 | 70.8 Kg | 1.8 Kg | 71.3 Kg | 2.3 Kg |
| Week 3 | 71.2 Kg | 1.9 Kg | 72 Kg | 3.8 Kg |
| Week 4 | 70.9 Kg | 2.0 Kg | 73.4 Kg | 4.3 Kg |
| Week 5 | 70.9 Kg | 2.1 Kg | 74.2 Kg | 5.4 Kg |
| Week 6 | 71.1 Kg | 2.2 Kg | 75.4 Kg | 5.5 Kg |

There was a clinically and statistically significant difference in weight gain during the trial. The combination group gained on average 2.2 (2.5) kg, as compared to 5.5 (2.7) kg in the olanzapine-placebo group (p<0.05). Significantly fewer patients in the combination group gained at least 7% of their initial weight, the cutoff for clinically significant weight gain (6 [20%] of 30 patients vs. 6 [46.1%] of 13 patients).

A previously conducted study using a similar study design, methods, duration of trial and comparable sample of first-episode predominantly drug-naive young schizophrenia patients treated with olanzapine and reboxetine (4 mg/day), showed that the reboxetine group gained 3.31 (2.73) kg as compared to the placebo group 4.91 (2.45) (p<0.01).

Similarly, betahistine was associated with a modest attenuation of weight gain in a small group (n=7) of olanzapine-treated first-episode schizophrenia patients: the betahistine group 3.86 kg versus the placebo group 5.03 kg.

Overall, the results of the systematic investigations of the weight-attenuating effect of reboxetine, betahistine and the combination of the two agents, suggest that the addition of betahistine to reboxetine resulted in a more pronounced effect (synergistic effect) on body weight in olanzapine-treated patients during the 6 week trial: reboxetine vs. betahistine vs. reboxetine/betahistine 3.31 kg vs. 3.86 kg vs. 2.21 kg.

Notably, there was a clinically important difference between the two groups in triglyceride levels after 6 weeks of the trial: while there was almost two-fold increase in triglyceride level in the olanzapine-placebo group (baseline: 98.0±40.1; endpoint: 117.6±58.5), a modest increase was noted in the olanzapine-combination group (baseline: 118.0±53.6; endpoint: 134.1±52.7). Decrease in triglyceride levels from baseline to endpoint was found in 8 of 19 (42.1%) of olanzapine +combination patients for whom two measurements were available, compared to only 1 of 7 (14.3%) olanzapine+placebo treated patients.

### Example 2

As in the previously conducted study, using a similar study design, methods, duration of trial and comparable sample of first-episode, predominantly drug-naive young schizophrenia patients were treated with olanzapine and reboxetine (4 mg/day), and showed that the reboxetine group gained 3.31 (2.73) kg as compared to the placebo group 4.91 (2.45) (p<0.01) [6].

Similarly, betahistine was associated with a modest attenuation of weight gain in a small group (n=7) of olanzapine-treated first-episode schizophrenia patients: the betahistine group gained 3.86 kg versus the placebo group 5.03 kg.

Overall, the results of the systematic investigations of the weight-attenuating effect of reboxetine, betahistine and the combination of the two agents, suggest that the addition of betahistine to reboxetine resulted in a more pronounced complementary effect on body weight in olanzapine-treated patients during the 6 week trial:

Weight gain: mean(SD)
Olanzapine+placebo 4.91 (2.45)kg (29 patients)
Olanzapine+reboxetine 3.31 (2.73)kg (31 patients)
Olanzapine+betahistine 3.86 (0.9)kg (7 patients)
Olanzapine+combination 2.10 (2.25)kg (17 patients)

Attenuation of weight-gain exerted by the combination was statistically significant versus placebo (p<0.01). There was a trend of a difference versus each of the components in favor of the combination, which was most probably accounted for by the small sample sizes. If treatment group include 30 patients each, a difference between the drug combination and its individual components would become statistically significant (p<0.05).

The combination of reboxetine and betahistine was well tolerated and no clinically significant side effects were revealed. Two patients complained on mild nausea and urinary retention probably related to the study combination.

In conclusion, the addition of betahistine to reboxetine and the use of this combination in olanzapine-treated patients prevented a substantial weight gain associated with olanzapine treatment. The combination was safe and well tolerated and did not counteract with olanzapine therapeutic effect. Hence, the combination between reboxetine and betahistine should be effective also in treatment of overweight and obesity in non-psychiatric population as well.

1-38. (canceled)

39. A composition for controlling weight-gain in a subject, the composition comprising at least one H₃-receptor antagonist and at least one norepinephrine reuptake inhibitor (NRI).

40. The composition according to claim 39, wherein said subject is obese.

41. A composition according to claim 39, wherein said controlling of weight-gain being achievable by suppression of appetite in said subject.

42. A composition according to claim 39 for reducing or maintaining plasma triglyceride levels in a subject, the composition comprising at least one H₃-receptor antagonist and at least one norepinephrine reuptake inhibitor (NRI).

43. The composition according to claim 39, wherein the subject is being treated with a drug inducing weight-gain.

44. The composition according to claim 43, wherein the drug inducing weight-gain is a psychiatric drug.

45. A composition for treating a psychiatric disease or disorder in a subject, without substantially inducing weight gain and/or without substantially increasing plasma triglyceride levels in said subject, the composition comprising at least one psychiatric drug, at least one H₃-receptor antagonist and at least one NRI.

46. The composition according to claim 45, wherein said psychiatric drug is selected to be effective in reducing at least one psychiatric symptom associated with a psychiatric disease or disorder, in a patient suffering therefrom or has a predisposition to suffer therefrom.

47. The composition according to claim 46, wherein said psychiatric drug is an atypical antipsychotic.

48. The composition according to claim 47, wherein the atypical antipsychotic is selected from the group consisting of amisulpride, aripiprazole, asenapine, blonanserin, clotiapine, clozapine, iloperidone, mosapramine, olanzapine, paliperidone, perospirone, quetiapine, remoxipride, risperidone, sertindole, sulpiride, ziprasidone and zotepine.

49. The composition according to claim 48, wherein the atypical antipsychotic is olanzapine.

50. The composition according to claim 39, wherein the subject has at least one eating disorder, said disorder being optionally selected from the group consisting of binge eating disorder (BED), Bulimia nervosa and Nocturnal Sleep Related Eating Disorder (NSRED).

51. The composition according to claim 39, wherein said H₃-receptor antagonist is selected from the group consisting of betahistine hydrochloride, betahistine mesylate, R-a-methyl histamine, methimepip, ciproxifan and thioperamide.

52. The composition according to claim 39, wherein the NRI is selected from the group consisting of serotonin-norepinephrine-dopamine reuptake inhibitors, selective norepinephrine reuptake inhibitors (SNRI), norepinephrine-dopamine reuptake inhibitors (NDRIs), serotonin-norepinephrine reuptake inhibitors (SNRIs), tricyclic antidepressants (TCAs), and tetracyclic antidepressants (TeCAs).

53. The composition according to claim 39, wherein the at least one H₃-receptor antagonist is betahistine and the at least one NRI is reboxetine.

54. A method for controlling weight gain a subject, the method comprising administering to said subject a combination of at least one H₃-receptor antagonist and at least one norepinephrine reuptake inhibitor (NRI).

55. A method according to claim 54, wherein the subject is obese, and said method being for weight reduction in said subject.

56. A method according to claim 54, for the suppression of appetite in a subject.

57. A method for treating a psychiatric disease or disorder in a subject, without substantially inducing weight-gain and/or without substantially increasing plasma triglyceride levels in said subject, the method comprising administering to said subject, a combination of at least one psychiatric drug, at least one H₃-receptor antagonist and at least one norepinephrine reuptake inhibitor (NRI).

58. The method according to claim 54, wherein said combination comprising betahistine and reboxetine.
